(19) United States
(12) Patent Application Publication
(10) Pub. No.: US 2017/0368264 A1
(43) Pub. Date: Dec. 28, 2017

(54) SYRINGE PLUNGER-STOPPER

(71) Applicant: APTAR STELMI SAS, Villepinte (FR)

(72) Inventors: Ghislain FOURNIER, La Rochelle (FR); Mickaël SWAL, Chauconin Neufmontiers (FR)

(73) Assignee: APTAR STELMI SAS, Villepinte (FR)

(21) Appl. No.: 15/546,307

(22) PCT Filed: Jan. 28, 2016
(86) PCT No.: PCT/FR2016/050173

§ 371 Date: Jul. 26, 2017

(30) Foreign Application Priority Data
Jan. 29, 2015 (FR) 1550678

(51) Int. Cl. A61M 5/315; A61L 31/04

(52) U.S. Cl. CPC A61M 5/31513; A61L 31/048; A61L 31/049

(57) ABSTRACT

A stopper/piston (510) having a cylindrical body (511) having a front axial end closed by a front wall (513). The hollow cylindrical body (511) includes an outside surface provided with a secondary sealing profile (610), the front wall (513) including a front axial outside surface (631) provided with a coating (635). The front wall (513) includes a primary sealing profile (630) on its radially-outer edge, the front wall (513) and the cylindrical body (511) is made of different materials, the hollow cylindrical body (511) being overmolded on the front wall (513). The front axial outside surface (631) of the front wall (513) includes a deformation profile (633) arranged radially inside the primary sealing profile (630), the deformation profile adapted to deform radially for insertion of the stopper/piston (510) into a syringe body.

The present invention relates to a syringe stopper/piston.

Stopper/pistons for syringes are well known in the prior art. Before the syringe is actuated, the stopper/piston performs the function of a stopper, isolating the fluid contained in the syringe body, and then during actuation, it transforms into a piston, pushing the fluid out of the syringe body, generally through a needle. Generally, the syringe device includes an actuator member, such as a piston rod, that co-operates with said stopper/piston so as to move it in the syringe body during actuation. The stopper/piston thus needs to guarantee sealing, and it is thus generally made of rubber or some other similar elastomer. A drawback with that type of material relates to the risks of interaction between the material of the stopper/piston and the fluid to be dispensed, in particular during the storage stage, where such interaction might possibly spoil said fluid. In order to limit the risks of interaction, it has been proposed to coat the front surface of the stopper/piston, i.e. the surface in contact with the fluid while storing and while dispensing the fluid, with an appropriate coating. In particular, it has thus been proposed to have a thin film of ethylene tetrafluoroethylene (ETFE) on the front surface of the stopper/piston. That technique makes it possible to limit the risks of interaction between the material of the stopper/piston and the fluid. However, applying said film makes the manufacturing method more complex, and requires a cutting zone on the stopper/piston after molding, which is to be avoided. Furthermore, the presence of the coating on the front surface stiffens said surface, and may thus make it more difficult to assemble and move the stopper/piston in the syringe body. More precisely, the presence of the front coating reduces the capacity of the front surface of the stopper/piston to deform, and may thus make it more difficult to insert it in the syringe body and to slide it therealong. There thus exists a problem of finding a good compromise between sealing that is sufficient for the stopper/piston once inserted in the syringe body, and a method of assembly and of actuation that is not too complicated or difficult.

In addition, co-operation between the stopper/piston and the actuator member, generally a piston rod, generally occurs by means of a screw thread that is provided in the inside surface of the stopper/piston and that co-operates with a corresponding thread that is formed on the leading end of the piston rod. That technique also complicates manufacture of the stopper/piston, in particular the unmolding stage, as a result of the large amount of back-drafts required in the mold for manufacturing the stopper/piston in order to mold the thread.

Documents WO 2006/021380, EP 1 180 377, EP 1 674 121, DE 100 06 560, WO 02/092312, JP 2001 190667, US 2007/219508, WO 2012/101669, EP 1 002 551, US 2014/062036, U.S. Pat. No. 5,735,825, and EP 2 803 378 describe prior-art devices.

Document WO 2015/052429 describes a syringe stopper/piston that is similar to the syringe stopper/piston of the present invention.

An object of the present invention is to provide a stopper/piston that does not have the above-mentioned drawbacks.

In particular, an object of the present invention is to provide a stopper/piston that guarantees complete sealing during storage and during actuation, while enabling manufacture, assembly, and actuation to be simplified and reliable.

Another object of the present invention is to provide a stopper/piston that is simpler and thus less costly to manufacture and to assemble, in particular in the process of molding said stopper/piston.

The present invention thus provides a stopper/piston comprising a cylindrical body having a front axial end that is closed by a front wall, said stopper/piston including an outside surface that is provided with at least one secondary sealing profile, said front wall including a front axial outside surface that is provided with a coating, advantageously a film made of ethylene tetrafluoroethylene, said front wall including a primary sealing profile on its radially-outer edge, said front wall and said cylindrical body being made of different materials, said hollow cylindrical body being overmolded on said front wall, said front axial outside surface of said front wall including a deformation profile that is arranged radially inside said primary sealing profile of said front wall, said deformation profile being adapted to deform radially so as to make it easier to insert and/or to slide said stopper/piston into a syringe body.

Advantageously, said cylindrical body is made of a rubber material, e.g. comprising one or more synthetic elastomeric polymers such as butyl rubber, chlorobutyl rubber, bromobutyl rubber, isobutylene and para-methylstyrene rubber copolymers, isoprene rubber, styrene butadiene rubber (SBR), nitrile butadiene rubber (NBR), ethylene propylene diene terpolymer (EPDM).

Advantageously, said material is associated with at least one accelerator and/or filler.

Advantageously, said front wall is made of a rubber material, e.g. comprising one or more synthetic elastomeric polymers such as butyl rubber, chlorobutyl rubber, bromobutyl rubber, isobutylene and para-methylstyrene rubber copolymers, isoprene rubber, SBR, NBR, EPDM.

Advantageously, said material is associated with at least one accelerator and/or filler.

Advantageously, said at least one accelerator and/or filler of said cylindrical body is different, in quantity and/or in composition, from said at least one accelerator and/or filler of said front wall.

Advantageously, said cylindrical body includes a secondary sealing profile, and said front wall includes a secondary sealing profile.

Advantageously, said secondary sealing profiles are radially-projecting beads.

Advantageously, said deformation profile is made in the shape of a peripheral groove that is formed in said front axial outside surface of said front wall, said groove advantageously being V-shaped or U-shaped.

Advantageously, said cylindrical body is hollow and defines an internal volume, said hollow cylindrical body having an open rear axial end that is axially remote from said front axial end that is closed by said front wall.

Advantageously, said internal volume co-operates with an actuator member of a syringe device.

Advantageously, on its inside surface, said hollow cylindrical body includes a screw thread that is adapted to be engaged on a thread of said actuator member.

Advantageously, said front axial outside surface is conical and defines a central axial tip.

Advantageously, said deformation profile is of rounded shape, such that said front axial outside surface of said front wall does not have any sharp angle.

These characteristics and advantages and others of the present invention appear more clearly from the following detailed description, given by way of non-limiting example, and with reference to the accompanying drawings, in which:

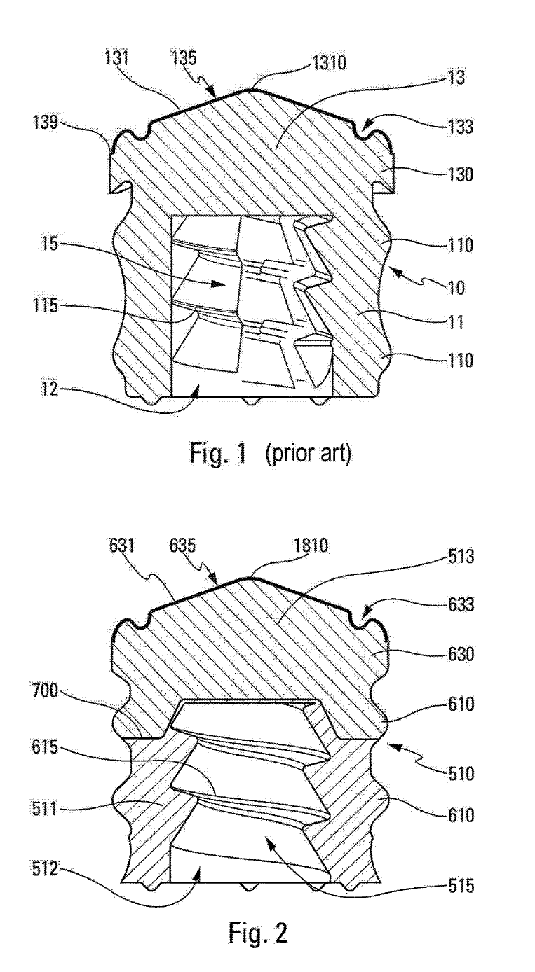

FIG. 1 is a diagrammatic section view of a stopper/piston of the prior art.

FIG. 2 is a view similar to the view in FIG. 1, showing a stopper/piston in an advantageous embodiment of the present invention.

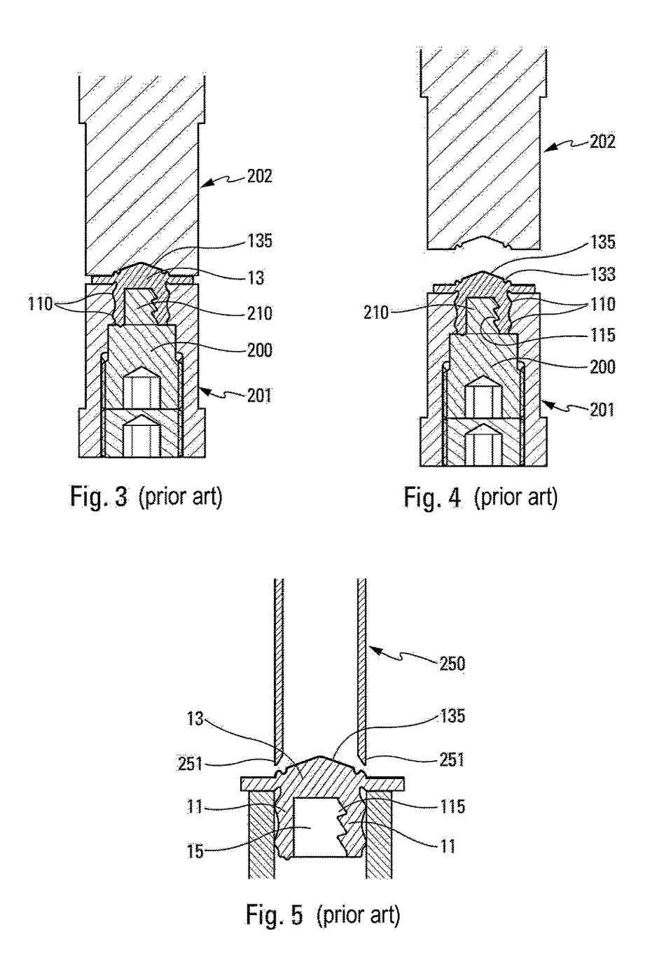

FIGS. 3 and 4 are diagrams showing the molding step of the prior-art method of manufacturing the stopper/piston of FIG. 1, with the mold shown closed and open respectively;

FIG. 5 is a diagram showing the cutting step of the prior art method of manufacturing the FIG. 1 stopper/piston; and

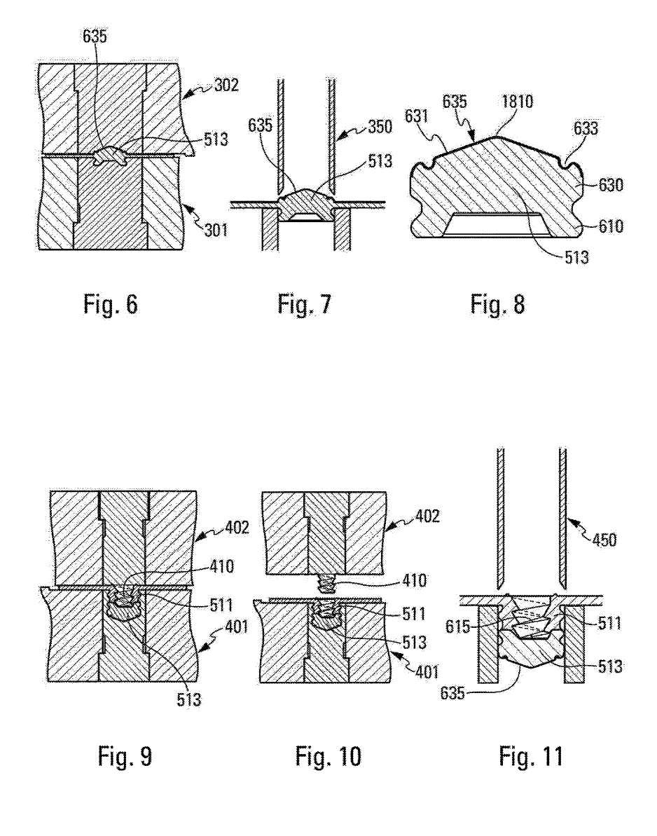

FIGS. 6 to 11 are diagrams showing the molding and cutting steps in an advantageous implementation of the method of the invention for manufacturing the FIG. 2 stopper/piston.

FIG. 1 shows a stopper/piston of the prior art, in particular as described in co-pending patent application WO 2015/052429.

The stopper/piston 10, molded as a single piece of elastomer, rubber, or any other appropriate material, comprises a cylindrical body 11 that is preferably hollow. The hollow cylindrical body includes an open rear axial end 12, and a front axial end that is closed by a front wall 13. Thus, this embodiment defines a stopper/piston 10 of shape that is generally cylindrical, with an internal volume 15 that is blind, i.e. open at one end and closed at the other end.

The front wall 13 defines a front axial outside surface 131 that is the surface that is in contact with the fluid when the stopper/piston 10 is assembled in a syringe body (not shown) for containing such a fluid. The front axial outside surface 131 is provided with a coating 135, advantageously a thin film made of ETFE, so as to limit interaction between said fluid and the material of said stopper/piston as much as possible. Other types of appropriate coating could also be envisaged, both with regard to the method of applying the coating and with regard to the material constituting said coating. The front axial outside surface 131 may be conical, defining an axial tip 1310, as can be seen in the FIG. 1 embodiment, but it could also be plane and substantially perpendicular to the central axis of the stopper/piston.

The lateral outside surface of the hollow cylindrical body 11 includes at least one sealing profile 110, and preferably a plurality of them that are generally made in the shape of radially-projecting beads. Preferably, and as shown in FIG. 1, the outside surface of the hollow cylindrical body 11 includes two sealing profiles 110 that are made in the shape of radially-projecting beads. The front wall 13 may also include a sealing profile 130 that is made on its radially-outer edge.

Said front axial outside surface 131 of said front wall 13 includes a deformation profile 133 that is adapted to deform so as to make it easier to insert the stopper/piston 10 into a syringe body. The deformation profile may be arranged radially inside said sealing profile 130, i.e. it is not necessarily arranged at the radially-outer edge of said front wall 13.

Advantageously, the deformation profile 133 may be made in the shape of a peripheral groove that is formed in said front axial outside surface 131. In the embodiment shown in FIG. 1, before being deformed, the groove is substantially U-shaped, but obviously it could also have some other shape, e.g. a V shape, a W shape, or any other appropriate shape that enables the front wall 13 to deform radially while the stopper/piston is being inserted.

Advantageously, said deformation profile 133 is of rounded shape, i.e. it does not define a sharp angle in said front axial outside surface 131 of said front wall 13. This is because such sharp angles might generate zones of weakness in the coating 135, not only while molding the stopper/piston, during which they might generate portions that become torn off, but also in use during which the coating 135 might crack or tear at said sharp angles, e.g. while compressing the stopper/piston when putting it in place in the syringe, with the consequent risk of said coating being discontinuous.

Advantageously, said coating 135 extends not only over said front axial outside surface 131, but also over at least a part of the outside cylindrical surface of said sealing profile 130. This makes it possible to guarantee the presence of a first cylindrical sealing zone between the inside of a syringe body and the coated portion of said sealing profile 130 of the stopper/piston 10. In particular, this improves sealing while high pressure is being exerted in the syringe. Specifically, a cylindrical contact zone between the syringe body and the coated zone of the stopper/piston 10 guarantees the absence of micro-wrinkles in the coating 135, and thus makes it possible to have a sealing zone with contact that is constant between the coating 135 of the stopper/piston and the syringe body.

In particular, the coating 135 extends until it reaches a shoulder 139 of said sealing profile 130. The shoulder 139 is generated by the method of manufacturing the FIG. 1 stopper/piston, as described in greater detail below with reference to FIGS. 3 to 5.

While inserting the stopper/piston 10 into a syringe body, the deformation profile 133 may deform radially, and this enables the sealing profile 130 of the front wall 13 to penetrate more easily inside the syringe body.

After inserting said stopper/piston 10 into the syringe body, the various sealing profiles, i.e. the sealing profile 130 of the front wall 13 and the sealing profiles 110 of the hollow cylindrical body 11 may relax a little, while remaining sufficiently constrained against the cylindrical wall of the syringe body, so as to guarantee complete sealing.

The presence of the deformation profile 133 in the front axial outside surface 131 of the front wall 13 makes insertion easier. Specifically, the presence of the coating 135 stiffens the material and thus generally makes it more difficult to insert. Provision of a deformation profile makes it possible to compensate for the increase in friction associated with the ETFE coating, and thus makes it possible to guarantee that the coated stopper/piston is inserted in more reliable and safe manner, as described above. In addition, the presence of the deformation profile 133 in the front axial outside surface 131 of the front wall 13 of the piston with coating makes it possible to have behavior that is identical to the behavior of a piston without coating, in particular with regard to the activation and sliding forces applied to the piston during actuation.

The internal volume 15 of the stopper/piston 10 may include a thread 115. The thread 115 may be continuous or discontinuous. In the variant with a discontinuous thread, a plurality of axial grooves, advantageously four grooves, may be provided so as to interrupt said thread 115.

FIGS. 3 to 5 show a machine for manufacturing the FIG. 1 stopper/piston, with a first mold portion 201 that is the bottom portion in FIGS. 3 and 4, and a second mold portion 202 that is the top portion. The first mold portion 201 is used to form the hollow cylindrical body 11, and includes a core 200 for this purpose. The core 200 includes an axial endpiece 210 that defines said internal volume 15 of said stopper/piston 10. This method of manufacturing the FIG. 1 stopper/piston is complex since it requires the first mold portion 201 and the axial endpiece 210 of the core 200 to have a plurality of back-drafts, so as to form firstly the internal thread 115 and secondly the sealing profiles 110. This makes unmolding more difficult.

FIG. 5 shows the step of cutting the stopper/piston. It should be observed that because of the coating 135, cutting cannot be performed while the material is taut, thereby generating a diameter of cut that is slightly greater than the diameter of the molded sealing profiles 110, and thereby generating said shoulder 139, as can be seen in FIG. 1. Thus, the first sealing zone formed by the sealing profile 130 occurs at the cutting zone, thereby generating a surface state that is not smooth, with consequent potential risks of infiltration and/or of sliding of the piston. Specifically, a piston having, as its first sealing zone, a cutting zone that includes micro-fluting (as a result of cutting) causes:
poorer sealing performance (micro-passages for the liquid via the fluting); and
poorer sliding as a result of the surface state and as a result of the diameter of the cut being greater than the diameter of the sealing profiles 110 made by molding, since the cutting cannot be performed under tension because of the stiffness of the coating 135.

In known manner, the stopper/piston 10 may also be coated entirely with silicone oil so as to enhance its sliding, in particular while it is being inserted into the syringe. In a variant, when it is desired to omit silicone oil, a parylene coating may alternatively be provided, likewise over the entire stopper/piston. In this configuration, the ETFE film on the front surface of the piston could be treated so as to provide grip for the parylene.

In the invention, the stopper/piston is made by overmolding two portions. FIG. 2 shows such a stopper/piston, and FIGS. 6 to 11 show a method of manufacturing said stopper/piston.

For the purposes of clarity, the elements of said stopper/piston and of said method of the invention that are similar to those of the prior art shown in FIGS. 1 and 3 to 5 are given the same numerical references plus 500.

Thus, in the invention, the stopper/piston 510 comprises a first molded portion that is formed of a front wall 513, and a second portion that is overmolded on said first molded portion and that is formed of a cylindrical body 511 that is preferably hollow. The line of separation 700 shows this two-part construction.

When the cylindrical body 511 is hollow, it includes an open rear axial end 512, and a front axial end that is closed by said front wall 513. Thus, this embodiment defines a stopper/piston 510 of shape that is generally cylindrical, with an internal volume 515 that is blind, i.e. open at one end and closed at the other end. The front wall 513 defines a front axial outside surface 631 that is the surface that is in contact with the fluid when the stopper/piston 510 is assembled in a syringe body (not shown) for containing such a fluid. The front axial outside surface 631 is provided with a coating 635, advantageously a thin film made of ETFE, so as to limit the interaction between said fluid and the material of said stopper/piston as much as possible. Other types of appropriate coating could also be envisaged, both with regard to the method of applying the coating and with regard to the material constituting said coating. The front axial outside surface 631 may be conical, defining an axial tip 1810, as can be seen in the FIG. 2 embodiment, but it could also be plane and substantially perpendicular to the central axis of the stopper/piston.

The front wall 513 includes a primary sealing profile 630 that is made on its radially-outer edge. The outside surface of the stopper/piston 510 further includes at least one secondary sealing profile 610, and preferably a plurality of them that are generally made in the shape of radially-projecting beads. Preferably, and as shown in FIG. 2, the outside surface of the stopper/piston 510 includes two secondary sealing profiles 610 that are made in the shape of radially-projecting beads. In the embodiment shown in FIG. 2, one of the secondary sealing profiles 610 is formed on said front portion 513 and the other on said hollow cylindrical body 511, but, in a variant, both secondary sealing profiles 610 could be formed on said hollow cylindrical body 511.

Said front axial outside surface 631 of said front wall 513 includes a deformation profile 633 that is adapted to deform so as to make it easier to insert the stopper/piston 510 into a syringe body. The deformation profile may be arranged radially inside said primary sealing profile 630, i.e. it is not necessarily arranged at the radially-outer edge of said front wall 513. Advantageously, the deformation profile 633 may be made in the shape of a peripheral groove that is formed in said front axial outside surface 631. In the embodiment shown in FIG. 2, before being deformed, the groove is substantially U-shaped, but obviously it could also have some other shape, e.g. a V shape, a W shape, or any other appropriate shape that enables the front wall 513 to deform radially while the stopper/piston is being inserted.

Advantageously, said deformation profile 633 is of rounded shape, i.e. it does not define a sharp angle in said front axial outside surface 631 of said front wall 513. This is because such sharp angles might generate zones of weakness in the coating 635, not only while molding the stopper/piston, during which they might generate portions that become torn off, but also in use during which the coating 635 might crack or tear at said sharp angles, e.g. while compressing the stopper/piston when putting it in place in the syringe, with the consequent risk of said coating being discontinuous.

Advantageously, said coating 635 extends not only over said front axial outside surface 631, but also over at least a part of the outside cylindrical surface of said sealing profile 630. This makes it possible to guarantee the presence of a first cylindrical sealing zone between the inside of a syringe body and the coated portion of said primary sealing profile 630 of the stopper/piston 510. In particular, this improves sealing while high pressure is being exerted in the syringe. Specifically, a cylindrical contact zone between the syringe body and the coated zone of the stopper/piston 510 guarantees the absence of micro-wrinkles in the coating 635, and thus makes it possible to have a sealing zone with contact that is constant between the coating 635 of the stopper/piston and the syringe body.

While inserting the stopper/piston 510 into a syringe body, the deformation profile 633 may deform radially, and this enables the primary sealing profile 630 of the front wall 513 to penetrate more easily inside the syringe body.

After inserting said stopper/piston 510 into the syringe body, the various sealing profiles, i.e. the primary sealing profile 630 and the secondary sealing profiles 610 may relax a little, while remaining sufficiently constrained against the cylindrical wall of the syringe body, so as to guarantee complete sealing.

The presence of the deformation profile 633 in the front axial outside surface 631 of the front wall 513 makes insertion easier. Specifically, the presence of the coating 635 stiffens the material and thus generally makes it more difficult to insert. Provision of a deformation profile makes it possible to compensate for the increase in friction associated with the ETFE coating, and thus makes it possible to guarantee that the coated stopper/piston is inserted in more reliable and safe manner, as described above. In addition, the presence of the deformation profile 633 in the front axial outside surface 631 of the front wall 513 of the piston with coating makes it possible to have behavior that is identical to the behavior of a piston without coating, in particular with regard to the activation and sliding forces applied to the piston during actuation.

The internal volume 515 of the stopper/piston 510 may include a thread 615. The thread 615 may be continuous or discontinuous. In the variant with a discontinuous thread, a plurality of axial grooves, advantageously four grooves, may be provided so as to interrupt said thread 615. In a variant, the stopper/piston may be solid, i.e. not including a hollow internal volume.

The stopper/piston 510 of the invention, made of two overmolded portions, presents the particular advantage of being formed from two different materials having respective properties that are optimized. Thus, mechanical properties (hardness, elasticity, elongation) make it possible to optimize the functional properties of the piston (sliding, sealing, machine handling, etc. . . . ), and chemical properties of the materials (level of extractables, etc. . . . ) make it possible to use a high purity material for the front wall 513 that is in contact with the film (1^{st} molding step), and to combine it with another material having optimized functional properties (in the 2^{nd} molding step).

Advantageously, both the cylindrical body 511 and the front wall 513 are made of rubber materials, e.g. comprising one or more synthetic elastomeric polymers such as butyl rubber (isobutylene and isoprene copolymer), chlorobutyl rubber (isobutylene and isoprene copolymer derived from the reaction of a butyl rubber with chlorine), bromobutyl rubber (isobutylene and isoprene copolymer derived from the reaction of a butyl rubber with bromine), isobutylene and para-methylstyrene rubber copolymers, isoprene rubber (synthetic polyisoprene), SBR (styrene and butadiene copolymer), NBR (acrylonitrile butadiene copolymer), EPDM (ethylene propylene diene terpolymer). These materials may be associated with various accelerators and/or fillers, making it possible to obtain the desired characteristics.

In the invention, said cylindrical body 511 and said front wall 513 are made of different materials. The rubber materials may be different. In a variant, the cylindrical body 511 may advantageously be made of a material that might be made up of the same rubber bases as the rubber bases of said front wall 513, but that are associated with one or more fillers and/or accelerators that differ in their quantities and/or compositions.

FIGS. 6 to 11 show the method of manufacturing the FIG. 2 stopper/piston, in an advantageous embodiment of the present invention.

FIG. 6 shows the front wall 513 and the coating 635 being molded in a first mold that conventionally comprises two mold portions 301, 302. Unmolding is easy, since said mold does not include any back-draft.

FIG. 7 shows said molded front wall being cut, in particular by means of a punch or of a die 350.

FIG. 8 shows the front wall 513 after molding and cutting, and before overmolding the hollow cylindrical body 511.

FIGS. 9 and 10 show the hollow cylindrical body 511 being overmolded on the front wall 513. The front wall 513 is arranged in a first mold portion 401 of a second mold, and said hollow cylindrical body 511 is overmolded around a core 410 of a second portion 402 of said second mold. Said core 410 defines said internal volume 515, and it thus advantageously includes an external thread that defines the internal thread 615 of said stopper/piston 510. Unmolding is simplified: the core is extracted (e.g. by deforming the molded material) by moving the top portion of the mold, then the sheet is unmolded (back-drafts defining the secondary sealing profiles 610 can be unmolded by radially deforming the stopper/piston inwards as a result of the hollow left by extracting the core). Thus, during this overmolding step, the zone where said molded front wall is cut by the cutting step shown in FIG. 7, is reshaped in said second mold, and, as a result, the diameter of the cutting zone is less than the diameter that was cut. As a result of placing said molded front wall in a second mold, it is thus possible to avoid a drawback of the prior art as described above.

FIG. 11 shows the final cutting step. This time, cutting may be performed in conventional manner on the rear of the stopper/piston, at the open rear axial end 512, since when the stopper/piston does not include coating 635 on its front axial outside surface 631. This makes it possible to have a material that is taut while cutting, thereby generating radial shrinkage of the cut zone relative to the secondary sealing profiles 610. This cutting step is conventionally performed by means of a punch or of a die 450.

In known manner, the stopper/piston 510 may also be coated entirely with silicone oil so as to enhance its sliding, in particular while it is being inserted into the syringe. In a variant, when it is desired to omit silicone oil, a parylene coating may alternatively be provided, likewise over the entire stopper/piston. In this configuration, the ETFE film on the front surface of the piston could be treated so as to provide grip for the parylene.

Although the present invention is described above with reference to a particular embodiment, naturally the present invention is not limited by that embodiment, and, on the contrary, any useful modification could be applied thereto by the person skilled in the art, without going beyond the ambit of the present invention, as defined by the accompanying claims.

1. A stopper/piston (510) comprising a cylindrical body (511) having a front axial end that is closed by a front wall (513), said stopper/piston (510) including an outside surface that is provided with at least one secondary sealing profile (610), said front wall (513) including a front axial outside surface (631) that is provided with a coating (635), advantageously a film made of ethylene tetrafluoroethylene, said front wall (513) including a primary sealing profile (630) on its radially-outer edge, the stopper/piston being characterized in that said front wall (513) and said cylindrical body (511) are made of different materials, said hollow cylindrical body (511) being overmolded on said front wall (513), and in that said front axial outside surface (631) of said front wall (513) includes a deformation profile (633) that is arranged radially inside said primary sealing profile (630) of said front wall (513), said deformation profile (633) being adapted to deform radially so as to make it easier to insert and/or to slide said stopper/piston (510) into a syringe body.

2. A stopper/piston according to claim 1, wherein said cylindrical body (511) is made of a rubber material, e.g. comprising one or more synthetic elastomeric polymers such as butyl rubber, chlorobutyl rubber, bromobutyl rubber, isobutylene and para-methylstyrene rubber copolymers, isoprene rubber, SBR, NBR, EPDM.

3. A stopper/piston according to claim 2, wherein said material is associated with at least one accelerator and/or filler.

4. A stopper/piston according to claim 1, wherein said front wall (513) is made of a rubber material, e.g. comprising one or more synthetic elastomeric polymers such as butyl rubber, chlorobutyl rubber, bromobutyl rubber, isobutylene and para-methylstyrene rubber copolymers, isoprene rubber, SBR, NBR, EPDM.

5. A stopper/piston according to claim 4, wherein said material is associated with at least one accelerator and/or filler.

6. A stopper/piston according to claim 3, wherein said at least one accelerator and/or filler of said cylindrical body (511) is different, in quantity and/or in composition, from said at least one accelerator and/or filler of said front wall (513).

7. A stopper/piston according claim 1, wherein said cylindrical body (511) includes a secondary sealing profile (610), and said front wall (513) includes a secondary sealing profile (610).

8. A stopper/piston according to claim 7, wherein said secondary sealing profiles (610) are radially-projecting beads.

9. A stopper/piston according to claim 1, wherein said deformation profile (633) is made in the shape of a peripheral groove that is formed in said front axial outside surface (631) of said front wall (513), said groove advantageously being V-shaped or U-shaped.

10. A stopper/piston according to claim 1, wherein said cylindrical body (511) is hollow and defines an internal volume (515), said hollow cylindrical body (511) having an open rear axial end (512) that is axially remote from said front axial end that is closed by said front wall (513).

11. A stopper/piston according to claim 10, wherein said internal volume (515) cooperates with an actuator member of a syringe device.

12. A stopper/piston according to claim 11, wherein, on its inside surface, said hollow cylindrical body (511) includes a screw thread (615) that is adapted to be engaged on a thread of said actuator member.

13. A stopper/piston according to claim 1, wherein said front axial outside surface (631) is conical and defines a central axial tip (1810)

14. A stopper/piston according to claim 1, wherein said deformation profile (633) is of rounded shape, such that said front axial outside surface (631) of said front wall (513) does not have any sharp angle.
